(19) United States
(12) Patent Application Publication
(10) Pub. No.: US 2025/0186519 A1
(43) Pub. Date: Jun. 12, 2025

(54) USE OF A PROBIOTIC COMPOSITION IN A METHOD OF TREATMENT OF MANIFESTATIONS OF SELF-REPORTED MENTAL EXHAUSTION, PREFERABLY WITH MANIFESTATIONS OF LOW ENERGY LEVELS

(71) Applicants: WINCLOVE HOLDING B.V., Amsterdam (NL); INSTITUT ALLERGOSAN PHARMAZEUTISCHE PRODUKTE FORSCHUNGS- UND VERTRIEBS GMBH, Graz (AT)

(72) Inventors: Anita FRAUWALLNER, Graz (DE); Sandra Johanna Maria Ten Bruggencate, Amsterdam (NL); Jacoline Gerritsen, Amsterdam (NL)

(21) Appl. No.: 18/840,816

(22) PCT Filed: Feb. 24, 2023
(86) PCT No.: PCT/EP2023/054728

§ 371 Date: Aug. 22, 2024

(30) Foreign Application Priority Data
Feb. 25, 2022 (EP) 22159035.9

(51) Int. Cl. A61K 35/747; A61K 31/405; A61K 31/4415; A61K 35/00; A61K 35/744; A61K 35/745; A61P 25/28

(52) U.S. Cl. CPC A61K 35/747; A61K 31/405; A61K 31/4415; A61K 35/744; A61K 35/745; A61P 25/28; A61K 2035/115

(57) ABSTRACT

A multispecies probiotic composition including at least 5 species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum for use in treatment of a self-reported mental-disorder condition of mental exhaustion in a human.

The invention relates to use of pro-biotic compositions in self-medication of human subjects that are suffering or believed to be suffering from self-reported mental exhaustion (synonym with mental fatigue) and possibly aggravated by various manifestations of low energy levels and lack of sleep. Ishii A, Tanaka M, Watanabe Y. (Neural mechanisms of mental fatigue. Rev Neurosci. 2014; 25 (4): 469-79) define mental fatigue as a decline in the ability and efficiency of mental and/or physical activities that is caused by excessive mental and/or physical activities.

Knowledge about symptoms and healthcare seeking decisions provides an arena for understanding the interface between the healthcare system and the population. Since the 1960s we have witnessed a series of studies exploring the prevalence of symptoms in subjects and the proportion of healthcare seeking subjects. That the proportion of subjects self-reporting a symptom is often much larger than the proportion of those subjects seeking professional medical treatment for that symptom was identified as “The Symptom Iceberg” for the first time in 1963 by J M Last (The Iceberg “Completing the clinical picture” in general practice. Lancet. 1963;7297 (2): 28-31) and operationally defined by Hannay in 1979 (The symptom iceberg. Br J Gen Pract. 2011; 61 (584): 224). The phenomenon depicts two parts—the “submerged (self-reported) part” encompassing most symptom experiences (and which subjects report at least to themselves) that are not brought to the attention of a general practitioner (GP) or the medical community as a whole and for which self-medication may be practiced, and the “surfaced part” symbolising the proportion of symptoms that are presented to a medical practitioner because medical treatment by a medical professional is sought. As reviewed by Elnegaard et al., (Self-reported symptoms and healthcare seeking in the general population-exploring “The Symptom Iceberg”, BMC Public Health. 2015; 15:685) studies have illustrated that the decision process in subjects regarding healthcare seeking for a symptom is complex and depends on a variety of different factors, which possibly differ among men and women. Elnegaard et al., (ibid) reported on the incidence of a set of 44 self-reported (sometimes also referred to as patient-reported) symptoms in a set of 100,000 randomly selected subjects and categorized these according to the frequency with which these symptoms let the patient contacted the general practitioner (GP). The largest proportion of GP contacts was observed for individuals reporting blood in the urine 73.2%, whereas 11.4% of individuals with increase in waist circumference reported contact with the GP.

According to the American Psychological Association (APA) Work and Well-being Survey of 2021 encompassing 1,501 U.S. adult workers, 79% of employees had experienced self-reported work-related stress in the month before the survey. In the survey, nearly 3 in 5 employees reported negative impacts of work-related stress, including lack of interest, motivation, or energy (26%) and lack of effort at work (19%). Meanwhile, 36% reported cognitive weariness, 32% reported mental exhaustion, and an astounding 44% reported physical fatigue—a 38% increase since 2019. This phenomenon has even been termed the “Other Pandemic”. The past few years have been wrought with both personal and collective stress. A global pandemic, social unrest and, most recently, a war in Europe have increasingly contributed to reports of mental exhaustion. Changes in employment, losses of loved ones, isolation and a constant barrage of negative news stories have left many feeling mentally drained and unsure of how to bounce back from this state of chronic tension.

As defined by the NHS, stress is the body's reaction to feeling threatened or under pressure. Stress, very common, can on the one hand motivate to help us achieve things in our daily life, and can help us meet the demands of home, work, and family life. But on the other hand, too much stress can affect our mood, our body, and our relationships-especially when it feels out of our control. It can make us feel anxious and irritable and affect our self-esteem. Experiencing a lot of stress over a long period of time can also lead to a feeling of mental exhaustion.

Care should be taken to distinguish people suffering from (self-reported) mental exhaustion from those suffering from (validly diagnosed) clinical depression. Clinical depression is defined as a mood disorder that causes a persistent feeling of sadness and loss of interest and diagnosed according to the International Statistical Classification of Diseases and Related Health Problems (ICD-10) guidelines (Sheehan et al., The Mini-International Neuropsychiatric Interview (M.I.N.I.): The Development and Validation of a Structured Diagnostic Psychiatric Interview for DSM-IV and ICD-10. J. Clin. Psychiatry 1998, 59 (Suppl. 20), 22-33) and additionally valid psychiatric diagnostic tools such as the Leiden Index of Depression, revisited (LEIDS-R), as for example described in US20180271919 and a recent (published after the priority date of this application) study by Kreuzer et al. (The PROVIT Study-Effects of Multispecies Probiotic Add-on Treatment on Metabolomics in Major Depressive Disorder-A Randomized, Placebo-Controlled Trial. Metabolites 2022, 12, 770.) The broadly used M.I.N.I. is a short, structured interview held by a medical professional that screens for several psychiatric disorders and drug use. M.I.N.I. is used by medical clinicians to diagnose clinical depression and is typically not used by laymen or-woman in self-reporting disorders. Also called major depressive disorder (MDD), clinical depression affects how you feel, think, and behave and can lead to a variety of severe emotional and physical problems.

US20180271919 and Kreuzer et al. (ibid) do not mention nor are directed at treating (self-reported) mental exhaustion. With mental exhaustion, most problems are related to work and personal adjustments made in relation to work or resting routines will typically help. However, with depression, such personal adjustments typically do not help, a causal link with stress is not present, and other treatments such as (psycho-) therapy or medication may be needed. Also, depression has certain symptoms that mental exhaustion doesn't usually exhibit, including low self-esteem, feelings of hopelessness and/or suicidal thoughts.

Fatigue can also be classified as physical or mental. Mental fatigue manifests as potentially impaired cognitive function and is one of the most significant causes of accidents in modern society. Recently, it has been shown that the neural mechanisms of mental fatigue related to cognitive task performance are more complex than previously thought and that mental fatigue is not caused only by impaired activity in task-related brain regions. There is accumulating evidence supporting the existence of mental facilitation and inhibition systems. These systems are involved in the neural mechanisms of mental fatigue, modulating the activity of task-related brain regions to regulate cognitive task performance. To put it simply, mental exhaustion can happen when your brain receives too much stimulation or must maintain an intense level of activity without rest.

One might notice mental exhaustion, sometimes called mental fatigue, if one frequently works or studies for long hours with few or no breaks, spends a lot of time each day dealing with overwhelming responsibilities, lives with mental health symptoms, devotes a lot of mental energy each day to thinking through problems, worries, or feel overwhelmed. It's not at all uncommon to feel physically fatigued from time to time, and the same holds true for mental exhaustion or fatigue. Still, lingering mental fatigue can affect one's ability to think, solve problems, or process and regulate emotions. Eventually, it can even lead to challenges in one's daily life and in relationships. “Mental” tends to refer to cognitive skills, like thinking, memory, decision making, and problem-solving. “Emotional,” on the other hand, has to do with feelings, including your ability to identify, process, and express them. Also, many have come to accept that feeling mentally tired, weak, or drained of any energy is the price to pay for a busy social life, personal life, and career. But fatigue doesn't have to be a daily fight. Many people, especially women, don't think of fatigue as something that they should seek medical attention for. They see it as something that is just a symptom of a busy life, but if you have a healthy lifestyle and are generally healthy, you shouldn't be plagued by fatigue. There is often an underlying issue when women tell they're constantly tired. Where physical fatigue results from muscle fatigue brought about by intense physical activity, mental fatigue results from prolonged periods of cognitive activity which impairs cognitive ability. Mental fatigue can manifest as sleepiness without the ability to solidly sleep and rest, lethargy, or directed attention fatigue (Marcora S M, Staiano W, Manning V. “Mental fatigue impairs physical performance in humans”. Journal of Applied Physiology. 106 (3): 857-864). Mental fatigue can also impair physical performance. (Martin K, Meeusen R, Thompson K G, Keegan R, Rattray B. “Mental Fatigue Impairs Endurance Performance: A Physiological Explanation”. Sports Med. 48 (9): 2041-2051).

Chronic stress may affect a person's psychological well-being because of reduced serotonin production. Only a small proportion of serotonin is produced in the liver, spleen and brain, the intestinal mucosa manages to provide a remarkable 95% of this messenger substance. The starting material here is 5-hydroxy-tryptophan (5-HTP), the precursor of which is L-tryptophan. With the help of vitamin B6 and magnesium, 5-HTP is converted to serotonin. When we are under stress, this pathway no longer works properly. The serotonin production is therefore insufficient, which may lead to mental illnesses. Recent studies are focusing on the importance of the intestinal microbiome in the bidirectional communication between the gut and the brain. The gut microbiome produces signaling molecules that have a significant impact on the regulation of brain function, as well as human emotions and behaviour, as indicated in new studies on the use of probiotics in both healthy young people and psychiatric patients. The concept of the gut-brain axis represents an important approach for the development of new therapy strategies for mental illnesses and at the same time helps to optimize the cognitive functions in these patients, resulting in an enormous improvement in the quality of life.

A probiotic is a living microorganism, which when present in adequate amounts may confer a health benefit to the host. Probiotic bacteria can be consumed in the form of commercially-produced dietary supplements such as probiotic powders, capsules, tablets or fortified dietary products. Since beneficial effects of probiotics on mental health have been shown, they are also referred to as “psychobiotics”. Throughout the literature, there appear to be two dominant hypotheses addressing possible mechanisms of action through which probiotics exert their effects on mood symptoms. Theories involve the regulation of serotonin metabolism as well as regulation of inflammatory markers. The first has been demonstrated by Wallace C., et al, “The effects of probiotics on depressive symptoms in humans: a systematic review” (Ann Gen Psychiatry. 2017; 16:14). Probiotics in the GI tract have been shown to affect levels of serotonin in blood and in the brain via the gut-brain axis, and vitamin B or milk-derived products given together with psychobiotics have by some identified to help affect those levels.

The second has been demonstrated by Reiter, A., et al “Interleukin-6 Gene Expression Changes after a 4-Week Intake of a Multispecies Probiotic in Major Depressive Disorder-Preliminary Results of the PROVIT Study” (Nutrients 2020, 12, 2575) performed a randomized, placebo-controlled, multispecies probiotics study in depressed individuals. The probiotic intervention group showed decreasing pro-inflammatory IL-6 gene expression levels while the placebo group showed increasing gene expression levels of IL-6.

Probiotics could also be a useful additional treatment to mental therapy. Ansare F., et al, “The effects of probiotics and prebiotics on mental disorders: a review on depression, anxiety, Alzheimer and autism spectrum disorders” (Curr Pharm Biotechnol. 2020; 21:555) indicates that probiotics can improve mental health and psychological function and can be offered as new medicines for common mental disorders. Clinicians recommend probiotics to their patients. Dolan et al (Probiotics and Disease: A Comprehensive Summary-Part 1, Mental and Neurological Health. Integr Med (Encinitas). 2016 October;15 (5): 46-58.) provide a literature review of disease-specific probiotic strains studied in published clinical trials. The strain of Lactobacillus plantarum JYLP-326 is reportedly used for preparing powder product for improving sleep (claimed), inhibiting anxiety and depressive symptoms, and relieving mental fatigue (CN 110 792 451 A). Lalitsuradej et al. (2019 16th ECTI-CON) provide a study of the effect of Lactobacillus paracasei HII01 on cortisol in fatigue subjects and deem further studies mandatory to reveal the health beneficial effect of this strain. Daisuke Sawada et al. (Journal of Functional Foods 57, 2019, 465-476) show that daily intake of Lactobacillus gasseri CP2305 relieves fatigue and stress-related symptoms in male university Ekiden runners. Addressing more directly the neuroendocrine imbalances in chronic fatigue syndrome are 2 studies that show that supplementation with certain Lactobacillus species indeed can reduce symptoms of anxiety (Rao A V, Bested A C, Beaulne T M, et al. A randomized, double-blind, placebo-controlled pilot study of a probiotic in emotional symptoms of chronic fatigue syndrome, whereas of other Lactobacillus strains or combinations thereof such effects are not known. Gut Pathogen. 2009; 1:6; O'Sullivan E, Barrett E, Grenham S, et al. Brain-derived neurotrophic factor (BDNF) is of interest because of its putative role in stress and psychiatric disorders. Sullivan et al studied BDNF expression in the hippocampus of maternally separated rats (Does Bifidobacterium breve 6330 alter BDNF levels? Benef. Microbes. 2011; 2 (3): 199) and while B. breve 6330 influenced BDNF in normal animals, it had no significant effect on BDNF in stressed animals which were maternally separated. Of other Bifidobacterium strains or combinations thereof, such opposing effects are not known. Galland (The gut microbiome and the brain. J Med Food. 2014; 17 (12): 1261-1272.) showed that supplementation with Bacteroides fragilis also decreased anxiety levels, whereas of other Bacteroides strains or combinations thereof such effects are not known.

### The Invention

The invention provides a probiotic composition for use in treatment of a self-reported mental-exhaustion related condition in a human. Therewith, the invention provides a self-medical use directed at the submerged part of the symptom iceberg (as discussed above); those conditions and symptoms that are generally not reported to a GP or other medical professional and which treatment depends foremost on self-medication. It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. As further demonstrated in FIG. 1, the effect of 2-weeks dietary supplementation of a probiotic as provided herein on the number of participants reporting improved self-reported feelings of mental exhaustion, such a composition, when tested for use in a method of treatment for two weeks in a large population registering self-reported manifestations of mental disorders (effective size n=9897) was found to double the proportion of participants who hardly reported mental exhaustion and showed benefit in reducing conditions of mental exhaustion by said probiotic composition. The invention also provides a method of treatment directed at said submerged part of the symptom iceberg; a method of treatment of those conditions and symptoms that are generally not reported to a GP or other medical professional and which treatment depends foremost on self-medication. In particular, the invention provides a method of treatment of a self-reported mental-exhaustion related condition in a human comprising providing a human with a composition, as demonstrated in FIG. 1, wherein said condition comprises mental exhaustion. It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. In a furthermore preferred embodiment, the invention provides a self-medical use of such a composition and method of treatment with said condition comprises low energy-level (see FIG. 2, the effect of 2-weeks dietary supplementation of a probiotic as provided herein on the number of participants reporting improved self-reported feelings of energy-level). It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. In a further embodiment, the invention also provides a method of treatment by self-medication by use of a composition according to the invention wherein the self-medicated subject more often is waking up feeling well rested (see FIG. 3: the effect of 2-weeks dietary supplementation of a probiotic as provided herein on the number of participants waking up feeling well rested). It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. In a further embodiment, the invention also provides a method of treatment by self-medication by use of a composition according to the invention wherein the self-medicated subject more often is having a good sleep (see FIG. 4: the effect of 2-weeks dietary supplementation of a probiotic as provided herein on the number of participants having a good sleep). It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. In a further embodiment, the invention also provides a method of treatment by self-medication by use of a composition according to the invention wherein the self-medicated subject more often can sleep through uninterrupted by laying or being awake (see FIG. 5: The effect of 2-weeks dietary supplementation of a probiotic as provided herein on the number of participants sleeping through uninterrupted by laying or being awake. It is preferred that said composition comprises Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. Participants (n=14503, final evaluation n=9897) were recruited at pharmacies and at doctor's offices, to self-report and fill in several questionnaires relating to mental resilience. In a further embodiment, it is preferred to provide said composition or supplement with tryptophan. In a yet further embodiment, it is preferred to provide said composition or supplement with a milk-derived product. In a yet further embodiment, it is preferred to provide said composition or supplement with a vitamin B product. In a further embodiment, it is preferred wherein oral supplementation of said composition may modulate serotonin blood-levels in a human.

In a further embodiment, the invention provides a probiotic composition for use in treatment of a-low-energy-level related condition in a human. As further demonstrated in FIG. 2, such a composition as provided herein led to almost twice as many participants that mostly or always felt full of energy. As further demonstrated in FIGS. 3 to 5, said composition is also capable to resolve said mental issues and nearly doubles the number of persons waking up feeling well rested, increases the number of people having a good sleep and increases the number of people that sleep through uninterrupted by laying or being awake by one third.

It is preferred that said composition for use in treatment of a mental-exhaustion related condition in a human and/or for use in treatment of a-low-energy-level related condition in a human and for use in improving self-reported sleep, such as in conditions of mental exhaustion or having to deal with self-reported overwhelming responsibilities, comprises a multispecies probiotic composition, preferably comprising at least one, more preferably two species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum, more preferably comprising at least one, more preferably two species selected from the group of comprising Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. In certain embodiments of the invention is preferred that intake of a composition as provided herein is accompanied by vitamin B or milk-derived products.

It is moreover preferred that said composition comprises at least three, preferably at least 4, more preferably at least 5, more preferably at least 6 species, most preferably at least 7 species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum.

In a preferred embodiment, the invention provides a composition for use in treatment of a self-reported mental disorder related condition, preferably for use in treatment of mental exhaustion, in a human comprising at least 6 species selected from the group of Lactobacillus acidophilus, Lactobacillus paracasei, Bifidobacterium lactis, Lactobacillus salivarius, Lactococcus lactis, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus paracasei, Bifidobacterium lactis, Lactobacillus salivarius, Lactococcus lactis, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus acidophilus, Bifidobacterium lactis, Lactobacillus salivarius, Lactococcus lactis, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus acidophilus, Lactobacillus paracasei, Lactobacillus salivarius, Lactococcus lactis, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus acidophilus, Lactobacillus paracasei, Bifidobacterium lactis, Lactococcus lactis, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus acidophilus, Lactobacillus paracasei, Bifidobacterium lactis, Lactobacillus salivarius, Lactobacillus plantarum and Bifidobacterium bifidum. The invention also provides a method of treatment of a mental-disorder related condition, preferably mental exhaustion, comprising providing a human with a composition of bacterial species according to above.

In a preferred embodiment, the invention provides a composition for use in treatment of a self-reported mental disorder related condition, preferably for use in treatment of mental exhaustion, in a human comprising at least 6 species selected from the group of Lactobacillus casei, Lactobacillus acidophilus, Lactobacillus paracasei, Bifidobacterium lactis, Lactobacillus salivarius, Lactococcus lactis and Lactobacillus plantarum. The invention also provides a method of treatment of a mental-disorder related condition, preferably mental exhaustion, comprising providing a human with a composition of bacterial species according to above.

Preferred is a composition for use in treatment of a self-reported mental disorder related condition in a human wherein oral supplementation of said composition may increase serotonin level in a human, comprising Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23. The invention also provides a method of treatment of a mental-disorder related condition, preferably mental exhaustion, comprising providing a human with a composition of bacterial species according to above.

Such a probiotic composition with demonstrated test result as shown in the figures herein and discussed already above is obtainable through Winclove and/or Allergosan. The probiotic supplement may be provided by the “Institute Allergosan,” Austria and/or Winclove B V, Amsterdam, Netherlands. As a commercially available dietary supplement it includes up to nine bacterial strains (species) with at least 7.5 billion organisms per 1 portion (3 g). Bacterial strains (species) are B. bifidum W23, B. lactis W51, B. lactis W52, L. acidophilus W22, L. casei W56, L. paracasei W20, L. plantarum W62, L. salivarius W24 and L. lactis W19. Such compositions as provide herein are preferably used for treatment of self-reported mental disorders and may be admixed with for example B-vitamins to enhance gut-brain activity.

### Figure Legends

FIG. 1: The effect of 2-weeks dietary supplementation of a probiotic as provided herein on the number of participants reporting improved self-reported feelings of mental exhaustion. Participants (n=9897) were recruited at pharmacies and at doctor's offices, to self-report and fill in several questionnaires relating to mental resilience.

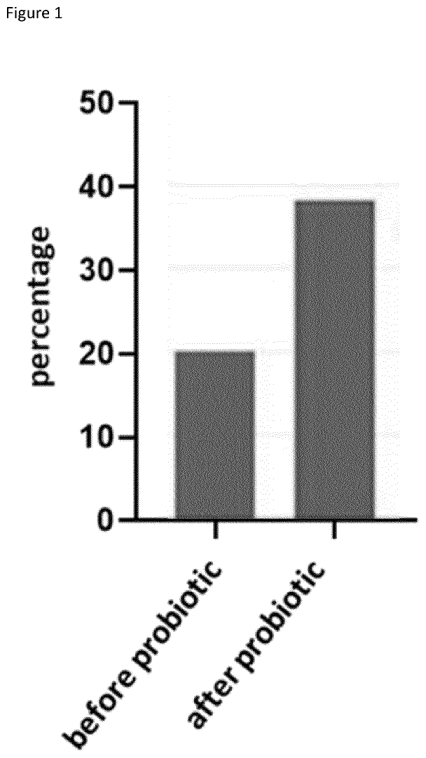

FIG. 2: The effect of 2-weeks dietary supplementation of a probiotic as provided herein on the number of participants reporting improved self-reported feelings of energy-level.

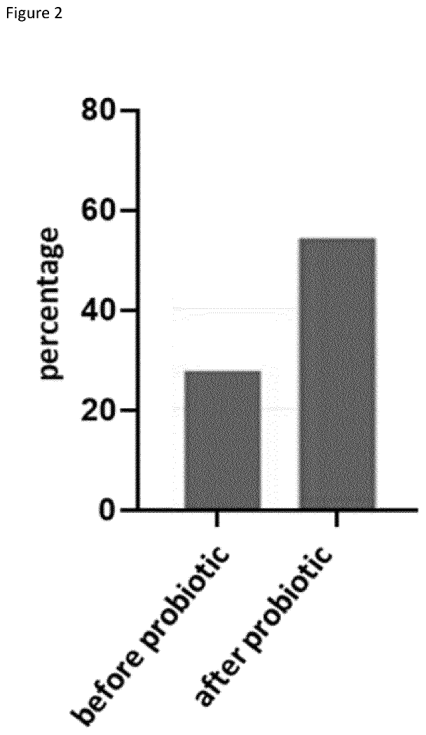

Participants (n=9897) were recruited at pharmacies and at doctor's offices, to self-report and fill in several questionnaires relating to mental resilience.

FIG. 3: The effect of 2-weeks dietary supplementation of a probiotic as provided herein on the number of participants waking up feeling well rested. Participants (n=9897) were recruited at pharmacies and at doctor's offices, to self-report and fill in several questionnaires relating to mental resilience.

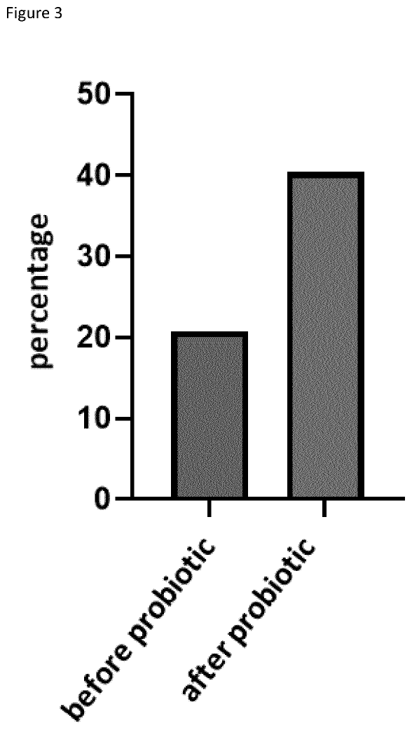

FIG. 4: The effect of 2-weeks dietary supplementation of a probiotic as provided herein on the number of participants having a good sleep. Participants (n=9897) were recruited at pharmacies and at doctor's offices, to self-report and fill in several questionnaires relating to mental resilience.

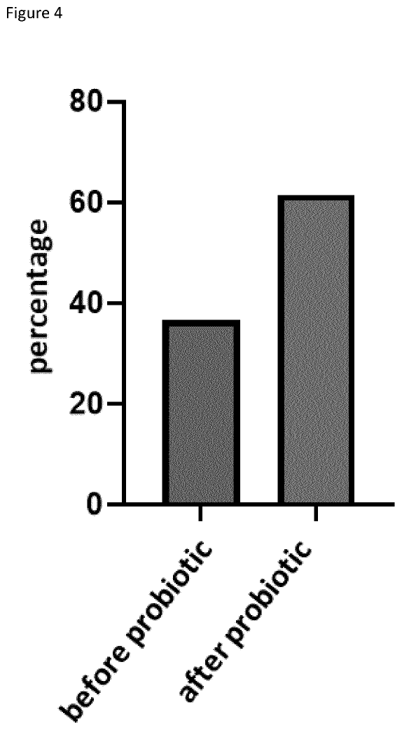

FIG. 5: The effect of 2-weeks dietary supplementation of a probiotic as provided herein on the number of participants sleeping through uninterrupted by laying or being awake. Participants (n=9897) were recruited at pharmacies and at doctor's offices, to self-report and fill in several questionnaires relating to mental resilience.

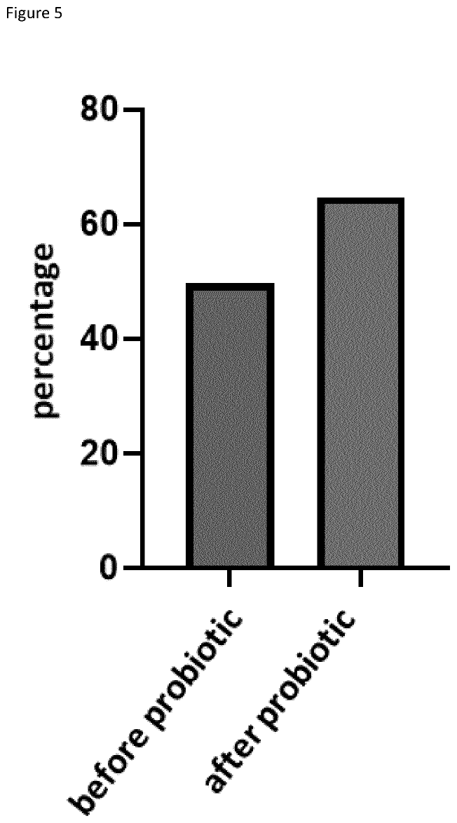

### DETAILED DESCRIPTION

Participants (n=14503, final evaluation n=9897) were recruited at pharmacies and at doctor's offices in Austria between May 2020-June 2021, to self-report and fill in several questionnaires relating to mental resilience. There were no restrictions on age or health indication, other than reported on the product's packaging.

The mean age in the combined population was 43 years with a standard deviation of 12 years. The primary indications for which probiotic supplementation was indicated was mental resilience during the corona pandemic.

Participants were asked to consume a probiotic composition with strains B. bifidum W23, B. lactis W51, B. lactis W52, L. acidophilus W22, L. casei W56, L. paracasei W20, L. plantarum W62, L. salivarius W24 and L. lactis W19, according to the user-instructions on the package for two weeks. Participants self-reported and filled in a health & well-being questionnaire at baseline and after 2-weeks of intake of said probiotic composition. Essential elements of said questionnaire are given at the following pages.

### Questionnaire

Please answer the following part A of the questionnaire before starting with your daily intake of the probiotic. Hereinafter, you find several statements. Please read each one and fill in the scale next to it, depending on how often you consider this statement to be true for your life now.

1-18. (canceled)

19. A multispecies probiotic composition comprising at least 5 species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum for use in treatment of a self-reported mental-disorder condition in a human, wherein said condition comprises mental exhaustion.

20. A composition according to claim 19 wherein said condition also comprises low energy-level.

21. A composition according to claim 19 wherein said condition also comprises a self-reported sleep-disorder.

22. A composition according to claim 19 comprising at least five species selected from the group of Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23.

23. A composition according to claim 19 comprising at least 6 species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum.

24. A composition according to claim 19 comprising at least 7 species selected from the group of Lactobacillus casei, Lactobacillus acidophilus, Lactobacillus paracasei, Bifidobacterium lactis, Lactobacillus salivarius, Lactococcus lactis, Lactobacillus plantarum and Bifidobacterium bifidum.

25. A composition according to claim 19 comprising Lactobacillus casei W56, Lactobacillus acidophilus W22, Lactobacillus paracasei W20, Bifidobacterium lactis W51, Lactobacillus salivarius W24, Lactococcus lactis W19, Bifidobacterium lactis W52, Lactobacillus plantarum W62 and Bifidobacterium bifidum W23.

26. A composition according to claim 19 supplemented with tryptophan.

27. A composition according to claim 19 supplemented with a milk-derived product.

28. A composition according to claim 19 supplemented with a vitamin B product.

29. A method of treatment of a mental-disorder related condition wherein said condition comprises mental exhaustion comprising the step of providing a human with a composition according to claim 19.

30. A method according to claim 29 wherein said condition comprises low energy-level.

31. A method according to claim 29 further comprising the step of providing said supplement with tryptophan.

32. A method according to claim 29 further comprising the step of providing said supplement with a milk-derived product.

33. A method according to claim 29 further comprising the step of providing said supplement with a vitamin B product.
